(19) United States
(12) Patent Application Publication
(10) Pub. No.: US 2016/0317583 A1
(43) Pub. Date: Nov. 3, 2016

(54) ENCAPSULATED STEM CELLS FOR THE TREATMENT OF INFLAMMATORY DISEASE

(71) Applicant: CytoStormRx LLC, New York, NY (US)

(72) Inventors: Michael B. Weiss, New York, NY (US); Martin H. Grumet, New York, NY (US)

(21) Appl. No.: 15/206,974

(22) Filed: Jul. 11, 2016

Related U.S. Application Data
(63) Continuation of application No. PCT/US15/62484, filed on Nov. 24, 2015.
(60) Provisional application No. 62/083,546, filed on Nov. 24, 2014.

(51) Int. Cl. A61K 35/28; A61K 38/20; A61K 31/191; A61K 9/50

(52) U.S. Cl. CPC A61K 35/28; A61K 9/50; A61K 38/204; A61K 31/191

(57) ABSTRACT

The present disclosure encompasses methods and compositions for the use of stem cells for the treatment of inflammatory diseases, which include, but are not limited, to sepsis. The disclosure also relates to a micro-encapsulation system for immobilizing stem cells, methods for delivery of encapsulated stem cells to a subject with inflammation, and use of the encapsulated stem cells as therapy for acute and chronic infections.

### CROSS-REFERENCE TO RELATED APPLICATIONS

This application is a continuation of PCT Application Serial No. PCT/US2015/062484, filed Nov. 24, 2015, which claims the benefit of priority of U.S. provisional application Ser. No. 62/083,546 filed on Nov. 24, 2014, the contents of each which are hereby incorporated by reference as if written herein in their entirety.

### BACKGROUND OF THE DISCLOSURE

Pathology in inflammatory diseases and disorders is caused by imbalances in inflammatory factors in a “cytokine storm”, which contributes to severe symptoms. Cytokine storm in sepsis reduces macrophage phagocytosis resulting in very high levels of toxic bacteria. Because a cytokine storm involves many inflammatory components and pathways, existing drugs are only partially effective even when used in combinations. Mesenchymal stem cells (MSCs) may control cytokine storms, which occur in many inflammatory diseases. The MSCs can be isolated from adult bone marrow and have the advantage of migrating to sites of inflammation within the body. MSCs can release anti-inflammatory factors, which can suppress a cytokine storm and reduce levels of bacteria, which are likely to have a therapeutic effect against inflammatory diseases in general, and particularly in sepsis.

MSCs delivered intravenously are safe but their efficacy in suppressing inflammation appears to be limited because they are cleared rapidly from the bloodstream and disappear from disease locations to which some of them they initially migrate. Although MSCs show limited rejection perhaps because of their inherent anti-inflammatory properties, they do not normally persist after injection in adult tissues except in bone marrow and a few other tissues that provide a supportive niche for MSC survival. To extend the life of MSCs in vivo, the inventor has encapsulated them in alginate microcapsules where they survive for much longer periods of time and can secrete their beneficial factors into surrounding regions within the body. The encapsulation has several advantages: 1) it allows MSCs to survive much longer in a subject; 2) it activates MSCs to secrete higher levels of factors that modulate inflammation locally and systemically; 3) it enables MSC capsules to be placed in specific regions where they can remain rather than migrate to distant locations where their fate is uncertain; 4) it allows use of lower doses of MSCs in disease models than reported previously to modify therapeutically relevant parameters; and 5) it protects the patient from escape of MSCs into the body where they might become tumorigenic, which is a concern when the they are genetically-modified to enhance their functions. The present disclosure relates to the discovery that MSCs respond in vivo to inflammatory diseases by altering their expression of immunomodulatory proteins and molecules. Thus, in accordance with an aspect of the present disclosure, provided herein are encapsulated stem cells, wherein the stem cells express an increased amount of at least one therapeutic protein or molecule in vivo compared to encapsulation of stem cells cultured in vitro.

### SUMMARY OF THE DISCLOSURE

The present disclosure provides a composition of encapsulated stem cells wherein said stem cells express an increased amount of at least one therapeutic protein or molecule in vivo compared to encapsulated mesenchymal stem cells cultured in vitro.

In one aspect of the disclosure the composition of the therapeutic protein is selected from the group consisting of epidermal growth factor (EGF), fibroblast growth factor (FGF), platelet-derived growth factor (PDGF), transforming growth factor-B (TGF-B), vascular endothelial growth factor (VEGF), hepatocyte growth factor (HGF), insulin-like growth factor-1 (IGF-1), angiopoietin-1 (Ang-1), keratinocyte growth factor (KGF), and stromal cell derived factor-1 (SDF-1) and combinations thereof.

In a second aspect of the disclosure the composition of the therapeutic protein is selected from the group of Tumor Necrosis Factor-Inducible Gene 6 Protein (TSG-6), Interleukin 4 (IL-4), Interleukin 5 (IL-5), Interleukin 6 (IL-6), Interleukin 10 (IL-10), Interleukin 33 (IL-33), Interleukin-1 receptor antagonist (IL-1RA), Galectin-1, Galectin-3, adiponectin, resolvin D1 (RvD1) or resolvin E1 (RvE1).

In a third aspect of the disclosure the composition of the therapeutic protein is selected from the group of prostaglandins, preferably prostaglandin E2 (PGE2).

In another aspect the composition of the therapeutic protein has a therapeutic effect to repair injured tissue caused by a disease, notably an inflammatory disease and/or to relieve symptoms of an inflammatory disease or disorder.

In yet another aspect the disease is an acute disease selected from the list of Sepsis, Ebola, Acute Lung Injury (ALI), (ARDS), Critical Limb Ischemia (CLI), Spinal Cord Injury (SCI), Traumatic Brain Injury (TBI), Acute Lung Injury (ALI), and Acute Respiratory Distress Syndrome (ARDS) or a chronic disease from the list of Inflammatory bowel disease (IBD), Crohn's disease, Rheumatoid arthritis (RA), Congestive Heart Failure, Amyotrophic Lateral Sclerosis (ALS), Diabetic Retinopathy (DR), Macular Degeneration (MD), Parkinson's Disease (PD), Multiple Sclerosis (MS), and Type 2 Diabetes.

One embodiment is an isolated population of encapsulated stem cells wherein such population in vivo exhibits secretion of a therapeutically relevant protein or molecule at a level at least 2 times greater than in vitro.

In a second embodiment, an isolated stem cell is modified in vitro to deliver a siRNA, miRNA, or dsRNA polynucleotide into a target cell comprising an exogeneous DNA sequence expressing the siRNA, miRNA, or dsRNA polynucleotide and which delivers the siRNA, miRNA, or dsRNA polynucleotide to the target cell via a microvesicle, exosome, or a cellular protrusion.

In a third embodiment, the isolated stem cell is placed in communication with a target cell under conditions suitable for transfer of the siRNA, miRNA, or dsRNA polynucleotide to the target cell via a microvesicle, exosome or a cellular protrusion.

In a fourth embodiment, the isolated stem cell is a mesenchymal stem cell (MSC) and the MSC delivers the exogenous DNA sequence or the siRNA, miRNA, or dsRNA sequence by a microvesicle, exosome, or a cellular protrusion.

In a fifth embodiment, the siRNA, miRNA, or dsRNA of the MSC is directed at a gene mediating a viral infection.

In another embodiment, the isolated mesenchymal stem cell is directed against the viral infection is caused by the Ebola virus.

In yet another embodiment, the isolated mesenchymal stem cell wherein the siRNA, miRNA, or dsRNA is directed at the NPC1 receptor gene.

In yet another embodiment the dose of encapsulated MSCs administered to a subject is less than 10,000,000 cells/kg weight of the subject.

In another embodiment of the disclosure, the MSCs are an expanded clonal or non-clonal population of mesenchymal stem cells.

In yet another embodiment of the disclosure, the micro-encapsulation system comprises an alginate microcapsule, wherein the micro-encapsulation system is capable of immobilizing the MSCs within an alginate microenvironment while sustaining molecular communication to relieve disease or its symptoms.

In another embodiment of the disclosure, the micro-encapsulation system comprising an alginate polymer wherein the microcapsule is highly permeable to serum albumin but not to immunoglobulin G (IgG).

In another embodiment of the disclosure, the micro-encapsulation system comprises an alginate polymer which has a concentration in the range from about 1.0% (w/v) to about 3% (w/v).

In another embodiment of the disclosure, the micro-encapsulation system comprises an alginate polymer which has a concentration of about 2.5% (w/v) wherein the microcapsule comprises additional sequential external surface coatings of poly-L-lysine.

In another embodiment is a method of treating a disease or its symptoms comprising administering to a subject suffering from a disease or its symptoms an effective amount of encapsulated mesenchymal stem cells to regulate an immune response in said subject to relieve disease or its symptoms.

In another embodiment the disclosure is an effective amount of encapsulated stem cells are administered to a subject by intraperitoneal (i.p.) injection, lymph node injection, thymus injection, spleen injection, intravenous injection, or combinations thereof.

In another embodiment the disclosure is an effective amount of encapsulated stem cells are administered by intraperitoneal injection within 1 day of diagnosis of a subject in need of treatment for sepsis.

In another embodiment the disclosure is an effective amount of encapsulated stem cells are administered by intraperitoneal injection within 1 day of diagnosis of a subject in need of treatment for sepsis combined with an effective amount of encapsulated mesenchymal stem cells administered 2-7 days later by intravenous or intraperitoneal injection.

In another embodiment the disclosure is an effective amount of stem cells encapsulated in alginate are administered to a subject by intravenous injection, intraperitoneal injection, lymph node injection, thymus injection, spleen injection, or combinations thereof.

In another embodiment the disclosure is a method of measuring the number of capsules injected in to each subject by measuring the capsules that were not injected and subtracting from the total number intended to be injected.

### BRIEF DESCRIPTION OF THE DRAWINGS

10× magnification images of the same field of encapsulated hMSCs (eMSC) with a diameter of about 400 μm. A single-plane projection of the fluorescent images obtained by confocal cross sectioning of a capsule after calcein/ethidium homodimer staining. FIG. 1A. Live cells. FIG. 1B. Dead cells. FIG. 1C. Differential interference contrast (DIC).

Bar graphs showing colony forming units (CFU)/ml of blood (FIG. 2A) and peritoneal lavage fluid (FIG. 2B) derived from CLP mice injected with 140,000 MSC/mouse or saline as a vehicle control. Data are presented as mean +/−SEM. *P, 0.05 and **P, 0.01 vs. saline vehicle (n=7 mice/group).

Bar graphs showing levels of IL-10 in FIG. 3A, IL-6 in FIG. 3B, TNFα in FIG. 3C, IL-1β in FIG. 3D, MCP-1 in FIG. 3E, and MCP-1 in FIG. 3F that were measured in blood derived from CLP mice injected with 140,000 MSC/mouse or saline. Data are presented as mean +/−SEM. *P, 0.05 and **P, 0.01 vs. saline vehicle (n=7 mice/group).

Bar graphs showing levels of IL-10 in FIG. 4A, IL-6 in FIG. 4B, TNFα in FIG. 4C, IL-1β in FIG. 4D, MCP-1 in FIG. 4E, and MCP-1 in FIG. 4F that were measured inperitoneal lavage fluid derived from CLP mice injected with 140,000 MSC/mouse or saline. Data are presented as mean +/−SEM. *P, 0.05 and **P, 0.01 vs. saline vehicle (n=7 mice/group).

FIG. 5. Shows the the number of capsules injected in each animal. Measurements were obtained for the total number of capsules that were not injected in each animal for the Encapsulated MSC (eMSC) Capsule Group and the Empty Capsule Group. (n=7 mice/group).

FIG. 6A. A Table showing the survival of CLP mice injected with 140,000 eMSC/mouse or saline (n=7 mice/group).

FIG. 6B. A table showing the number of live eMSC/capsule and the % of the live cells for microcapsules analyzed after 0-2 days of incubation in media in vitro, or recovered from septic mice the day after they were injected.

FIG. 7. Shows average levels of IL-6 secretion from eMSC microcapsules recovered from septic mice (ex vivo) or incubated only in vitro without activation (dotted line) or with LPS activation (solid line). The average of levels from mice #1, #2, #4, #5, and #6 were calculated.

FIG. 8. Shows levels of PGE2 secretion from eMSC incubated in vitro without activation or with LPS activation.

### DETAILED DESCRIPTION OF THE DISCLOSURE

The present disclosure provides the use of MSCs for the treatment of inflammatory diseases, which include, but are not limited, to sepsis.

In one broad aspect the present disclosure provides a method for using MSCs, which have the capacity to modulate immune responses and change the expression of different immunomodulatory factors in inflammatory diseases.

In another aspect of the disclosure, the regulatory properties of MSCs are immunosuppressive, reduce levels of bacteria and restore immune homeostasis in response to inflammation.

In another embodiment of this aspect, the disclosure provides MSCs, which modulate inflammation and promote tissue protection and/or repair. An MSC responds to pro-inflammatory cytokines by releasing anti-inflammatory cytokines, which can limit a cytokine storm and bacterial infection.

In another embodiment of this aspect, the disclosure provides an isolated cell population of MSCs encapsulated within an alginate polymer microenvironment.

In another aspect the present disclosure provides a micro-encapsulation system comprising an alginate polymer, wherein the system is capable of immobilizing mesenchymal stromal cells (MSCs) within an alginate microenvironment while sustaining molecular communication, wherein the encapsulated MSCs are capable of sustaining the MSC viability for a pre-determined amount of time.

In another aspect the present disclosure provides a method for promoting tissue protection, repair or treatment for inflammatory diseases or conditions in a subject, comprising administering to the subject an effective dose of MSCs encapsulated within an alginate polymer microenvironment, wherein the encapsulated MSCs are capable of surviving within said microenvironment for 3 months or longer.

In another aspect the present disclosure provides a kit. The kit MSCs encapsulated within an alginate polymer microenvironment, wherein the encapsulated MSCs cells are capable of surviving within said microenvironment for 3 months or longer.

In another aspect, the present disclosure sought to determine if alginate encapsulated MSCs could promote tissue repair and attenuate inflammation. The data described herein demonstrate that the microencapsulation platform can increase MSC secretion patterns in vivo which influence their ability to control a cytokine storm and bacterial infection, and that this function is also dependent upon encapsulation parameters. Furthermore, encapsulated MSCs in the presence of pro-inflammatory stimuli in vitro, can be induced to secrete these factors at increased rates. Through an in vivo model of sepsis, the inventor has demonstrated that encapsulated MSCs can mitigate expression of inflammatory factors, which reduce the effects during a cytokine storm, and reduces levels of bacteria in vivo.

The present disclosure, in one aspect, sought to investigate the feasibility of using the scalable and controllable alginate microenvironment culture system to induce MSCs to attenuate inflammation and levels of bacteria in vivo by injecting a lower dose of MSC than reported previously.

Alginate, a biocompatible copolymer of mannuronic and guluronic acid, has been used for many cell and tissue engineering applications, including, to encapsulate a suspension of MSC.

In a preferred embodiment of this aspect, the alginate polymer has a concentration guluronic acid of ˜60%.

In another preferred embodiment of this aspect, the alginate polymer has a concentration in the range from about 1.0% (w/v) to about 3% (w/v).

In a more preferred embodiment of this aspect, the alginate polymer has a concentration of about 2.5% (w/v).

In another preferred embodiment of this aspect, the alginate polymer has a concentration of about 2.5% (w/v) wherein the microcapsule comprises an additional sequential external surface coating of poly-L-lysine.

In another preferred embodiment of this aspect, microcapsule comprises a divalent cation from the group of calcium or barium or a mixture of calcium and barium to crosslink the alginate polymer into a microcapsule.

In another aspect the present disclosure provides a method for treating inflammatory diseases or conditions in a subject, comprising administering to the subject an effective dose of MSCs encapsulated within an alginate polymer microenvironment, wherein the dose of MSCs is less than 10 million cells/kg.

In one embodiment of this aspect, the inflammatory disease or condition is treated by delivering an effective dose of alginate encapsulated MSCs systemically.

In another embodiment of this aspect, the inflammatory disease or condition is treated by delivering an effective dose of alginate encapsulated MSCs in or adjacent to the site of inflammation.

In a preferred embodiment, the subject is a mammal.

In more preferred embodiment, the subject is a human and the MSCs are human MSCs (hMSCs). By taking advantage of the permeability of the alginate microcapsules to albumin but not to IgG and the use of low doses of MSC, the present inventor demonstrated a method to regulate the immune response to inflammation and bacterial clearance in vivo. In addition, MSCs can promote optimal MSC immune regulatory function in vivo, which can be achieved if 1) an effective delivery vehicle is designed, 2) sustained viability is established, 3) migratory capacity is controlled, 4) the location of the cells is defined and 5) tumor formation is suppressed. The present inventor has developed an MSC alginate polymer micro-encapsulation approach that addresses each of these criteria and has the potential for in vivo implantation. This approach will provide a controllable method for culturing and implanting MSCs and has the potential for ultimate translation into the clinic for treatment of acute and chronic infections, diseases or disorders. Thus, one objective of the present disclosure is to use alginate MSC encapsulation to (a) develop an immobilization platform for controlled delivery of anti-inflammatory MSCs for systemic treatment of inflammation, and/or (b) provide extended survival of MSCs for the purpose of controlling a cytokine storm.

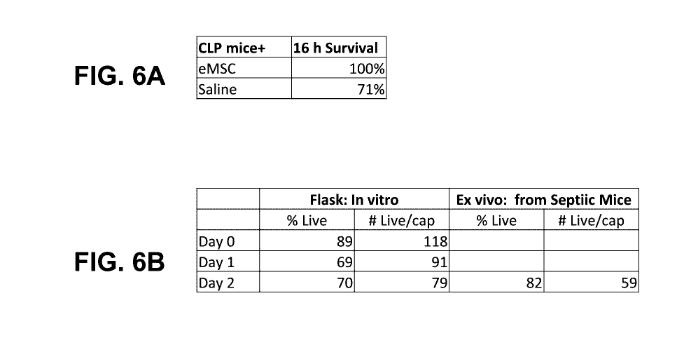

In order to circumvent various potential problems discussed above, this disclosure provides an alginate microencapsulation system as a vehicle for MSC delivery. Results show that in the absence of differentiation factor supplementation prior to injection in to a subject, the alginate microenvironment can be optimized to 1) support elevated secretion of anti-inflammatory mediators, 2) augment the immune-suppressive MSC phenotype over time and 3) induce secretion of at least one therapeutically relevant protein or molecule in vivo at a level at least 2 times greater than in vitro. Finally, using an in vivo model of sepsis, the disclosure has demonstrated that encapsulated MSCs can mitigate in blood and in peritoneum levels of bacteria by greater than 100-fold, and levels of inflammatory cytokines. This dramatic reduction in bacteria was unexpected given that prior experiments obtained much less potent reductions in bacteria with IP injection of 25-fold higher numbers of free human MSC compared to human ADSC. More comparable reductions in bacterial titers in blood and peritoneum were obtained with IP injection of 7-fold higher levels of mouse MSC in mouse sepsis (Hall et al., 2013). The relatively weak efficacy of free human MSC after IV injection can be attributed at least in part to the rapid loss of free cells by comparison to encapsulated cells (more than 50% survived, FIG. 6B), and the relative incompatibility of human MSC transplants in mouse (xenogeneic). Efficacy was improved with allogeneic (mouse in mouse) vs. xenogeneic transplants, but higher free doses may be required by comparison to encapsulated cells, because of rapid clearance of free MSC in contrast to robust survival in microcapsules.

These studies provide that alginate micro-encapsulation can be used as cell-derived molecular delivery systems with sustained and long-term function for the treatment of various tissue pathologies and acute and chronic diseases.

An important aspect of the present disclosure is that the alginate encapsulated MSCs of the present disclosure have tissue protective and anti-inflammatory properties, which are controlled via secreted products from the encapsulated MSCs or modulation of immune cells to increase bacterial clearance, and which may assist in reducing secondary consequences of traumatic injury or disease states. The capsules of the present disclosure are designed for in vivo injection for treatment of various conditions, including but not limited to Sepsis, Ebola, Acute Lung Injury (ALI), (ARDS), Critical Limb Ischemia (CLI), Spinal Cord Injury (SCI), Traumatic Brain Injury (TBI), Acute Lung Injury (ALI), and Acute Respiratory Distress Syndrome (ARDS), Inflammatory bowel disease (IBD), Crohn's disease, Rheumatoid arthritis (RA), Congestive Heart Failure, Amyotrophic Lateral Sclerosis (ALS), Diabetic Retinopathy (DR), Macular Degeneration (MD), Parkinson's Disease (PD), Multiple Sclerosis (MS), and Type 2 Diabetes.

The present disclosure has wide applications, including but not limited to 1) protecting endangered cells without the need for exogenous and expensive cytokines and growth factors, and 2) inducing and controlling secretion of anti-inflammatory and regenerative mediators to attenuate inflammation systemically and induce healing for a variety of in vivo applications, for both of which the present disclosure provides at least proof of concept. MSCs are known to exhibit anti-inflammatory responses when introduced to pro-inflammatory signals. However, direct contact between transplanted cells and the host may induce unfavorable immunological reactions that are diminished or eliminated by encapsulating MSCs and thereby preventing direct contact with the host. This also allows the use of non-autologous MSCs in a patient, and circumvents the delay required to collect and process autologous cells for individual patients. The pores in the alginate are sufficiently large to allow proteins such as albumin and small molecules to pass between the encapsulated cells and host, thus allowing the transplanted MSCs to be activated by soluble pro-inflammatory signals and release anti-inflammatory molecules. However, the pores are small enough to limit movement of immunoglobulin G (IgG) across the capsule protecting the cells from host antibodies. MSCs have been found to mediate inflammation and promote tissue repair through the secretion of a variety of soluble mediators with a wide array of physiological effects.

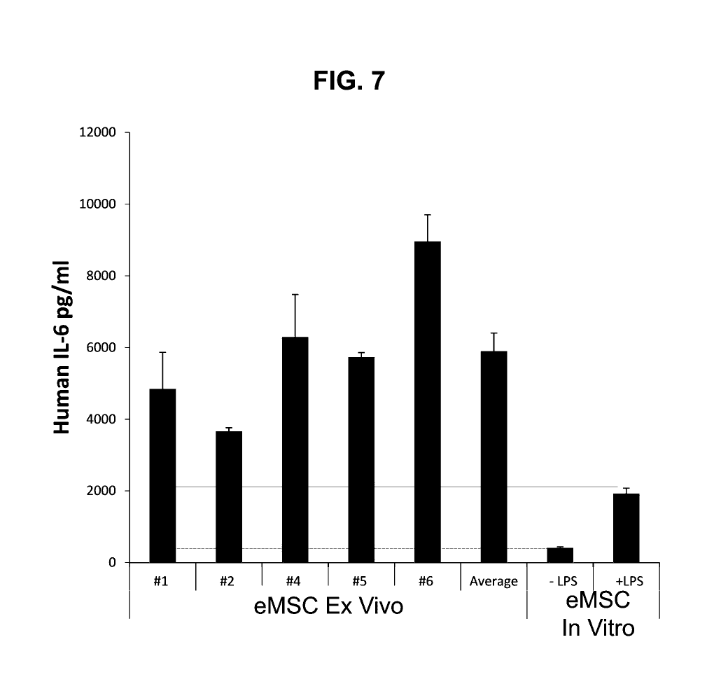

Therefore, the secretion profiles of encapsulated MSCs were analyzed to evaluate whether this characteristic was supported by the capsule platform. Evaluation of IL-6, which is an early indicator of MSC activation, indicated that it was secreted by encapsulated MSC ex vivo (recovered from septic mice) at greater than 10 times that secreted by encapsulated MSCs in vitro and at greater than 2 times that from encapsulated MSCs activated with LPS in vitro. The super-activation of encapsulated MSC in vivo to release higher levels of IL-6 than the maximal level of activation by LPS in vitro was unexpected (FIG. 7). This may have contributed to the robust reductions in bacteria in blood and peritoneal lavage by comparison to treatment after IP injection of other human MSCs.

The inventor here has also studied activation in encapsulated MSCs by measuring secretion of prostaglandin E2 (PGE2). Secretion of PGE2 was upregulated in encapsulated MSC when treated with LPS. PGE2 is known to play a critical role in mitigating the activation of M1 pro-inflammatory macrophages and promoting the M2 anti-inflammatory macrophage phenotype. This data suggests that activation of encapsulated MSCs may promote secretion of PGE2 and the shift of macrophages to the M2 phenotype. The M2 phenotype of activated macrophages is known to enhance phagocytosis of bacteria, which may lower levels of bacteria in sepsis.

The inventor has also found that a dose of non-autologous MSC in microcapsules (<6 million cells/kg), which is lower than that administered previously to a subject with CLP induced sepsis ˜40 million cells/kg), mitigated levels of bacteria and cytokine levels in blood and peritoneum. This suggests that fewer MSCs may be needed to achieve therapeutic benefits in vivo than previously reported wherein the MSC are super activated. Overall the data here support the fact that encapsulated MSCs may be used as immune-modulatory bio-reactors in vivo.

Encapsulation parameters were identified to maximize survival and enhance MSC protein secretion in a disease. In addition, we demonstrated that encapsulated MSCs attenuate levels of bacteria and cytokines, which, in vivo, could promote tissue protection. The immobilization system developed here should circumvent many of the drawbacks in current MSC administration platforms and at the same time may serve to augment MSC tissue protective behavior.

Thus, this disclosure proves, inter alia: 1) the alginate microenvironment can support MSC survival in a disease environment; 2) the alginate microenvironment increases MSC protein secretion in a disease environment; 3) within the alginate microcapsule, MSCs secrete immunomodulatory mediators, and encapsulated MSCs respond to pro-inflammatory stimuli by mitigating a cytokine storm systemically; 4) levels of bacteria in an infection can be attenuated by encapsulated MSCs locally and systemically; and 5) encapsulated MSCs are effective at a lower dose than reported previously.

### Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element

The term “about” will be understood by persons of ordinary skill in the art and will vary to some extent with respect to the context in which it is used.

As used herein the term “autologous” is meant to refer to any material derived from the same individual.

As used herein, the term “mesenchymal stem cells” or “MSCs” is to refer to a cell derived from bone marrow (reviewed in Prockop, 1997), peripheral blood {Kuznetsov et al., 2001), adipose tissue (Guilak et al., 2004), umbilical cord blood (Rosada et al., 2003), synovial membranes (De Bari et al., 2001), and periodontal ligament (Seo et al., 2005), embryonic yolk sac, placenta, umbilical cord, skin, and blood (U.S. Pat. Nos. 5,486,359 and 7,153,500), fat, and synovial fluid. MSC can also be derived from mesenchymal precursor cells called (MPC) (Psaltis et al. 2010) or multipotent adult progenitor cells (MAPCs) (U.S. Pat. Nos. 8,075,881 and 7,015,037). MSCs are characterized by their ability to adhere to plastic tissue culture surfaces (Friedenstein et al.; reviewed in Owen & Fricdenstein, 1988), and by being an effective feeder layers for hematopoietic stem cells (Eaves et al., 2001). In addition, MSCs can be differentiated both in culture and in vivo into osteoblasts, chondrocytes and adipocytes, and serve as progenitors for mesenchymal cell lineages including bone cartilage, ligament, tendon, adipose, muscle, cardiac tissue, stroma, dermis, and other connective tissues. (See U.S. Pat. Nos. 6,387,369 and 7,101,704.). Mesenchymal stem cells (MSCs) may be purified using methods known in the art (Wakitani et al 1995; Fukuda and Yuasa, 2006; Woodbury et al. 2000; Deng et al. 2000 Kim et al 2006; Maresehi et al. 2006; Krampera et al. 2007).

The term “growth factor,” as used herein, refers to a substance that is involved in cell differentiation and growth. The term is meant to include any regulator substance in morphogenesis.

The term “cytokine” is small protein released by cells that has a specific effect on the interactions between cells, on communications between cells or on the behavior of cells. Some cytokines promote inflammation such as tumor necrosis factor (TNFα) while others inhibit inflammation and promote repair and remodeling such as Interleukin (IL-10).

The term “cytokine storm” is an immune response gone awry and an inflammatory response flaring out of control.

The term “sepsis” is a potentially life-threatening complication of an infection. Sepsis occurs when chemicals released into the bloodstream to fight the infection trigger inflammatory responses throughout the body. This inflammation can trigger a cascade of changes that can damage multiple organ systems, causing them to fail. If sepsis progresses to septic shock, blood pressure drops dramatically, which may lead to death.

The term “septic shock” is a medical condition as a result of severe infection and sepsis, though the microbe may be systemic or localized to a particular site. It can cause multiple organ dysfunction syndrome (formerly known as multiple organ failure) and death.

A “subject” of diagnosis or treatment is a cell or a mammal, including a human. Non-human animals subject to diagnosis or treatment include, for example, simians, murines, guinea pigs, canines, such as dogs, leporids, such as rabbits, livestock, such as bovine or porcine, sport animals, and pets.

An “effective amount” is an amount sufficient to effect beneficial or desired results. An effective amount can be administered in one or more administrations, applications or dosages and can be empirically determined by those of skill in the art.

“RNA interference” (RNAi) refers to sequence-specific or gene specific suppression of gene expression (protein synthesis) that is mediated by short interfering RNA (siRNA).“Short interfering RNA” (siRNA) refers to double stranded RNA molecules (dsRNA), generally, from about 10 to about 30 nucleotides in length that are capable of mediating RNA interference (RNAi), or 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, or 29 nucleotides in length. As used herein, the term siRNA includes short hairpin RNAs (shRNAs). A siRNA directed to a gene or the mRNA of a gene may be a siRNA that recognizes the mRNA of the gene and directs a RNA-induced silencing complex (RISC) to the mRNA, leading to degradation of the mRNA. A siRNA directed to a gene or the mRNA of a gene may also be a siRNA that recognizes the mRNA and inhibits translation of the mRNA. “Double stranded RNA” (dsRNA) refer to double stranded RNA molecules that may be of any length and maybe cleaved intracellularly into smaller RNA molecules, such as siRNA. In cells that have a competent interferon response longer dsRNA, such as those longer than about 30 base pair in length, may trigger the interferon response. In other cells that do not have a competent interferon response, dsRNA may be used to trigger specific RNAi.A siRNA can be designed following procedures known in the art. See, e.g., Dykxhoorn, D. M. and Lieberman, J. (2006) “Running Interference: Prospects and Obstacles to Using Small Interfering RNAs as Small Molecule Drugs,” Annu. Rev. Biomed. Eng. 8:377-402; Dykxhoom, D. M. et al.(2006) “The silent treatment: siRNAs as small molecule drugs,” Gene Therapy, 13:541-52; Aagaard, L. and Rossi, J. J.(2007) “RNAi therapeutics: Principles, prospects and challenges,” Adv. Drug Delivery Rev. 59:75-86; de Fougerolles, A. et al. (2007) “Interfering With disease: a progress report on siRNA-based therapeutics,” Nature Reviews Drug Discovery 6:443-53; Krueger, U. et al. (2007) “Insights into effective RNAi gained from large-scale siRNA validation screening,” Oligonucleotides 17:237-250; US. Patent Application Publication No.: 2008/0188430; and US. Patent Application Publication No.: 2008/0249055.

Delivery of siRNA to a mesenchymal stem cell to generate the cell of this disclosure can be made with methods known in the art. See, e.g., Dykxhorn, D. M. and Lieberman, J. (2006) “Running Interference: Prospects and Obstacles to Using Small Interfering RNAs as Small Molecule Drugs,”Annu. Rev. Biomed. Eng. 8:377-402; Dykxhorn, D. M. et al. (2006) “The silent treatment: siRNAs as small molecule drugs,” Gene Therapy, 13:541-52; Aagaard, L. and Rossi, J. J. (2007) “RNAi therapeutics: Principles, prospects and challenges,” Adv. Drug Delivery Rev. 59:75-86; de Fougerolles, A. et al. (2007) “Interfering With disease: a progress report on siRNA-based therapeutics,” Nature Reviews Drug Discovery 6:443-53; Krueger, U. et al. (2007) “Insights into effective RNAi gained from large-scale siRNA validation screening,” Oligonucleotides 17:237-250; US. Patent Application Publication No.: 2008/0188430; and US. Patent Application Publication No.: 2008/0249055.

A siRNA may be chemically modified to increase its stability and safety. See, e.g Dykxhorn, D. M. and Lieberman, J. (2006) “Running Interference: Prospects and Obstacles to Using Small Interfering RNAs as Small Molecule Drugs,” Annu. Rev. Biomed. Eng. 8:377-402 and US Patent Application Publication No.: 2008/0249055.

MicroRNA or miRNA are single-stranded RNA molecules of 21-23 nucleotides in length, which regulate gene expression. miRNAs are encoded by genes from whose DNA they are transcribed but miRNAs are not translated into protein (non-coding RNA); instead each primary transcript (apri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

A siRNA vector, dsRNA vector or miRNA vector as used herein, refers to a plasmid or viral vector comprising a promoter regulating expression of the RNA. “siRNA promoters” or promoters that regulate expression of siRNA, dsRNA, or miRNA are known in the art, e.g., a U6 promoter as described in Miyagishi and Taira (2002) Nature Biotech. 20:497-500, and a H1 promoter as described in Brummelkamp et al. (2002) Science 296:550-3.

Mesenchymal stem cells (MSCs). Multipotent stem/stromal cells (MSCs) are also referred to in the art as bone marrow-, adipose-, umbilical cord-, and placental derived mesenchymal stem cells, and bone marrow-, adipose-, umbilical cord-, and placental-derived stromal cells. MSCs can be isolated using methods known in the art, e.g., from bone marrow, umbilical cord blood, adipose tissue, placental tissue, based on their adherence to tissue culture plastic. For example, MSCs can be isolated from commercially available bone marrow aspirates (Texas A&M University). MSCs also have a unique ability to reproducibly give rise to adipocytes, osteoblasts, and chondrocytes in vitro (Pittenger et al., Science, 284:143-147, 1999). MSC can also be derived from mesenchymal precursor cells called (MPC) or multipotent adult progenitor cells (MAPCs).

Mesenchymal stem cells (MSCs) from adult bone marrow have favorable properties for treating sepsis. MSC preferentially home to damaged tissue and may have therapeutic potential. They have been found to be safe and effective in clinical trials to facilitate engraftment in acute graft-versus-host disease as well as to repair tissue damage in inflammatory/degenerative disorders, in liver and inflammatory bowel diseases. MSCs favorably modulate the immune response to reduce lung injury, while maintaining host immune-competence and also facilitating lung regeneration and repair by immuno-modulating activated macrophages. Intravenous injection of bone marrow MSC can beneficially modulate a rapid response of the host immune system to sepsis and improve survival in animals. MSC secreted factors interact with circulating and tissue monocytes and macrophages and reprogram them. MSC treatment decreased the amounts of circulating IL-10 and IL-6, which are associated with poorer outcomes in human sepsis. This reduces harm caused by unbridled immune responses to the host tissue. Thus, human MSC have been found to be safe in clinical trials and effective in reducing the cytokine storm in several conditions including sepsis. Human MSC rapidly migrate to sites of injury and become activated to shift the milieu from a more pro- to a more anti-inflammatory/reparative state. Activation of MSC by pro-inflammatory factors including TNFα induces secretion of anti-inflammatory factors to reduce macrophage secretion of pro-inflammatory factors including TNFα, IL-1B, IP-10, MIP-1α. This reflects a reprogramming of macrophages from a pro-inflammatory M1 phenotype to an M2 anti-inflammatory/reparative phenotype. Thus, intravenous injection of human MSCs are safe and can modulate cytokine storms in various human diseases. One of the consequences of a cytokine storm is the breakdown in barrier function of the endothelial monolayer in the capillary bed in damaged tissues, allowing the release of protein-rich plasma and some leukocytes from the blood. MSCs produce various factors, like Ang-1, VEGF, HGF, EGF, PDGF, FGF, KGF and TGF-β, which directly affect endothelial cells. These paracrine trophic factors are potentially important in maintaining endothelial integrity and promoting angiogenesis through their ability to regulate endothelial cell proliferation and extracellular matrix production, reduce endothelial permeability or prevent interactions between leukocytes and endothelial cells.

Immunosuppressive properties of MSCs. Damaged tissues are always accompanied by infiltration of immune cells. Inflammation triggers the production of high levels of cytokines, chemokines and adhesion molecules in immune cells, including TNFα, IL-6, IL-1, CXCR3 ligands, CCR5 ligands, ICAM-1 and VCAM-1. These molecules induce the accumulation of immune cells, and in the presence of injected MSCs, high concentrations of NO (in murine) or depletion of tryptophan (in human) leads to the inhibition of immune cells. Other immunosuppressive factors such as IL-10, TSG6, IL-6, LIF, IL-1RA, PGE2, HO-1, truncated CCL2 and PGE2 could also affect immune cell activation, proliferation and functions The multitude of paracrine factors produced by MSCs, which provoke tissue-resident progenitor cells or other relevant cells to suppress inflammation and initiate tissue repair, may explain the beneficial effects of the transient survival of injected MSCs on tissue repair in a host, even in the absence of local MSC engraftment in the host.

Immunomodulatory activity of MSCs in the Liver. MSC-derived molecules reverse fulminant hepatic failure suggesting that much of the immunomodulatory activity of MSC resides in their secreted factors. These results suggested that factors secreted by MSC in response to pro-inflammatory factors (e.g. LPS, TNFα) were effective in shifting macrophages from a pro-inflammatory M1 phenotype to an M2 anti-inflammatory/reparative phenotype. Encapsulation of human bone marrow MSC is alginate microcapsules altered the MSC secretome and further treatment with TNFα yielded a more robust response that shifted macrophages from a pro-inflammatory M1 to an M2 anti-inflammatory/reparative phenotype both in vitro and in vivo. Thus, MSC when encapsulated in alginate can be segregated from the host to prolong their survival and prevent unwanted differentiation in the host while providing secreted factors that are immunomodulatory. The immunomodulatory factors released form MSC have been found to be effective in converting M1 to M2 macrophages in spinal cord contusion and in hepatocellular death and regeneration in vitro and in vivo.

Ebola. The Ebola virus may be acquired upon contact with blood or other bodily fluids of an infected human or other animal. Blood samples are tested for viral antibodies, viral RNA, or the virus itself to confirm the diagnosis. Laboratory testing with real-time polymerase chain reaction (PCR) is sensitive and specific and can return results within hours; it is now becoming more widely available in the affected areas. Thus, it is feasible to diagnose Ebola infections relatively early. Efforts to help those who are infected are supportive and include giving either oral rehydration therapy (slightly sweet and salty water to drink) or intravenous fluids. This supportive care improves outcomes. The disease has a high risk of death, killing between 25% and 90% of those infected with the virus (average is 50%).

No specific treatment for the disease is yet available but a range of blood, immunological and drug therapies are under development to neutralize the virus. Outbreak control requires a coordinated series of medical services, along with a certain level of community engagement. The necessary medical services required include rapid detection and contact tracing, quick access to appropriate laboratory services, proper management of those who are infected, and proper disposal of the dead. Prevention includes wearing proper protective clothing and washing hands when around a person with the disease. Samples of bodily fluids and tissues from people with the disease should be handled with special caution.

Ebola virus infection induces secretion of abnormal levels of cytokines into blood creating a “cytokine storm”. In fatal cases very high levels of the pro-inflammatory cytokines including TNF-α, IL-6, and IL-8, which are secreted by activated macrophages were found, as well as very low concentrations of the T cell cytokines, IL-2, IL-3, IL-4, IL-5, IL-9, and IL-13, which are anti-inflammatory. Ebola survivors were characterized by a transient release in plasma of (IL-1β), IL-6, (TNFα), macrophage inflammatory protein-1a (MIP-1α) and MIP-1β early in the disease, followed by circulation of IL-1 receptor antagonist (IL-1RA) towards the end of the symptomatic phase and after recovery. Thus, the presences of IL-1β and of elevated concentrations of IL-6 in plasma during the symptomatic phase have been proposed as markers of non-fatal infection. In addition, persistent increase in TNFα is associated with fatal Ebola infections.

Sustained siRNA production from human MSCs can be used to treat acute and chronic infections, notably to treat Ebola virus. Another embodiment of the present disclosure is to use human mesenchymal stem cells (MSCs) as safe delivery vehicles to knock down levels of the Niemanne Pick Disease receptor (NPC1). Human MSC engineered to produce anti-NPC siRNA can directly transfer enough RNA interfering molecules into cells in vitro to achieve significant reduction in levels of the NPC receptor protein. The transfer occurs through direct cell-to-cell transfer of siRNA or through exosome transfer.

The present disclosure is described more fully by way of the following, non-limiting examples.

### EXAMPLES

### Example 1

### MSC Cell Culture

hMSCs were purchased from Texas A&M University. Cells were grown in Alpha-MEM supplemented with 10% MSCs qualified FCS, 2 ng/ml L-glut and 1 ng/ml bFGF (complete medium). MSCs are seeded at 5,000 cells/cm² in falcon flasks and media was changed every four days.

### Example 2

### Micro-Encapsulation of MSC in Microcapsules

Alginate solutions (2.5%) were prepared by dissolving 2.5 g of sodium alginate (Pronova UP LVG, G Content: >60%, FMC Biopolymer Novamatrix) in 100 mL of Alpha-MEM, using a heated magnetic stir plate at a temperature of 65° C. The solution was then filtered using a 45 μm syringe filter (Fisher Scientific, Pittsburg, Pa.). To encapsulate cells in microcapsules, 0.9 mL of the 2.5% alginate solution was mixed with 0.1 mL aliquot of MSC suspension with a density of 6×10⁷ cells/mL to yield a final cell density of 6×10⁶ cells/mL and transferred to a 10 mL syringe and connected to a syringe pump (KD Scientific, MA). Alginate microcapsules were generated using an electrostatic bead generator using a PE-00940 needle (Nisco, Zurich, Switzerland) at a flow rate of 5 mL/h, and an applied voltage of 6.4 kV. The microcapsules were extruded into a 200 mL bath of CaCl₂ (100 mM), containing 145 mM NaCl, and 10 mM MOPS (all from Sigma-Aldrich) and left to polymerize for 10 min at room temperature. The microcapsules settled to the bottom of the tube and the solution was replaced with 0.05% (w/v) poly-L-Lysine (Sigma Aldrich, USA) in PBS and incubated for 10 minutes. The poly-L-lysine solution was removed, and replaced with HEPES solution to wash the microcapsules. The HEPES was removed and the microcapsules (FIG. 1A) were re-suspended in 5 mL of complete medium.

### Example 3

### Assay for Viability of MSC in Microcapsules In Vitro

MSC viability in the capsules was assessed using a calcein and ethidium homodimer assay (Molecular Probes, USA) (Maguire et al., 2007). Microcapsules were imaged in a confocal microscope (Zeiss 510 LSM) to acquire cross-section images of the alginate capsules at 6.38 μm intervals (FIG. 1A, FIG. 1B). The cross-sections were then compiled into a single plane image for quantification. Live cells in (green) and dead cells in red, were converted to black and white images in FIG. 1A and FIG. 1B, respectively. Live and dead cells were counted using the cell-counter plugin on ImageJ software and the percent of live cells/capsule was the number live/(total live+dead cells).

### Example 4

### Assay for Size of Microcapsules

Bright field images of capsules (FIG. 1C) were used to measure diameters using Image J image analysis software and averages were calculated.

### Example 5

### Assay for Efficiency and Reproducibility of Capsule Injections

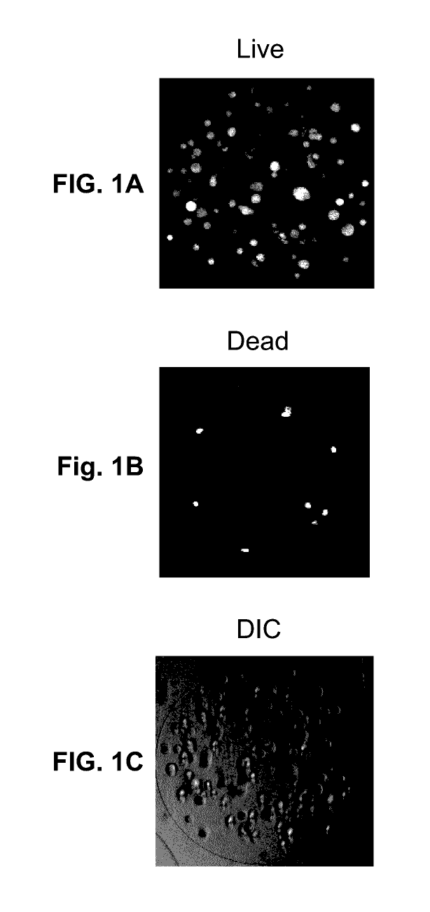

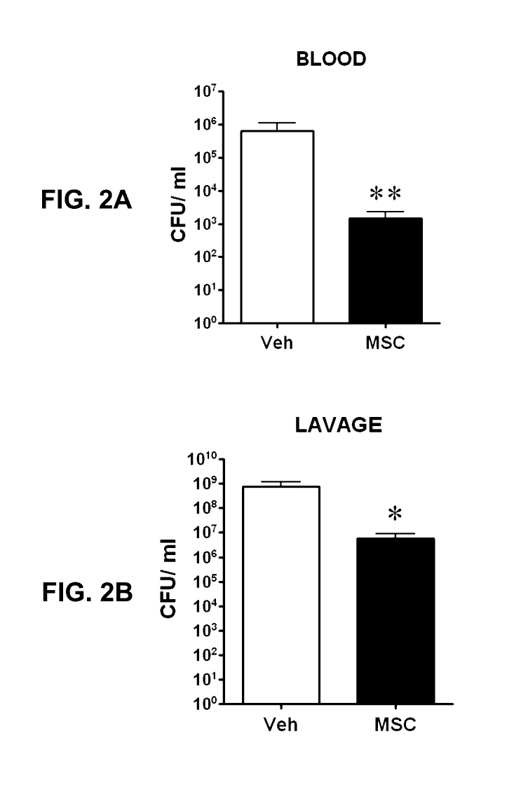

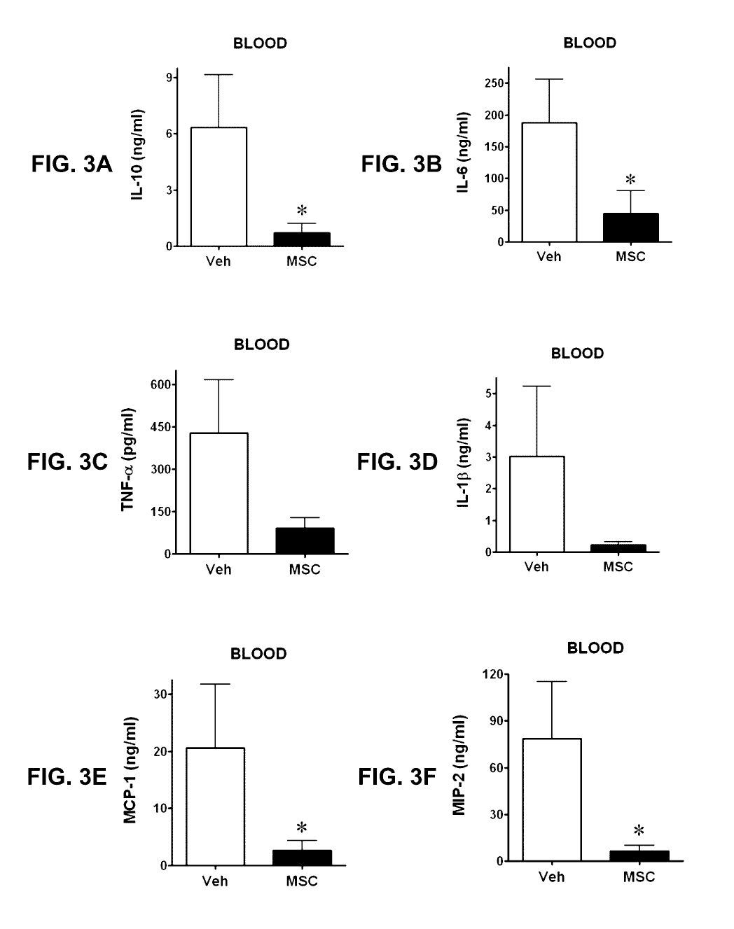

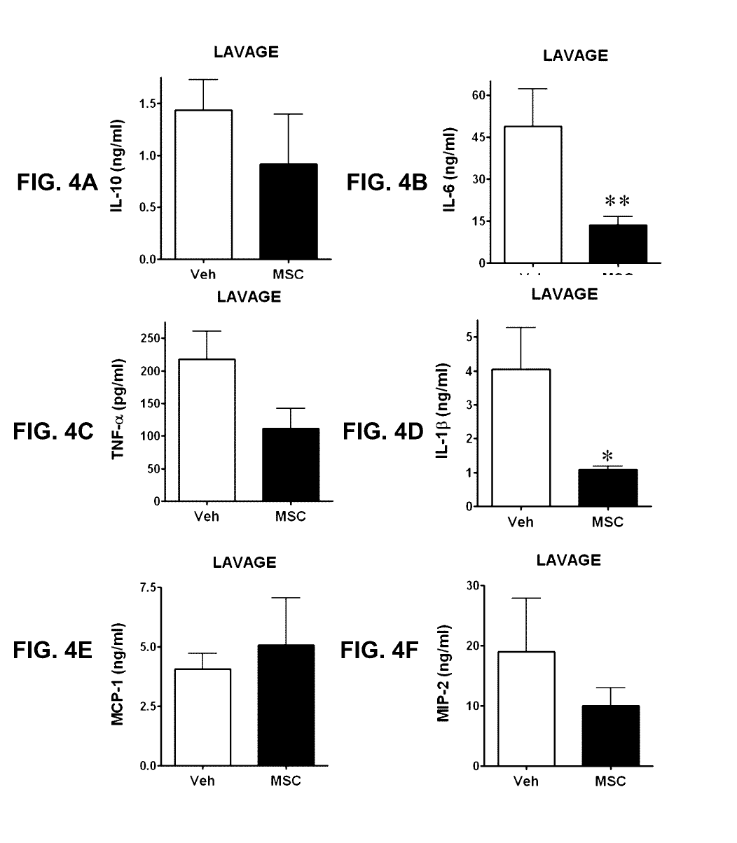

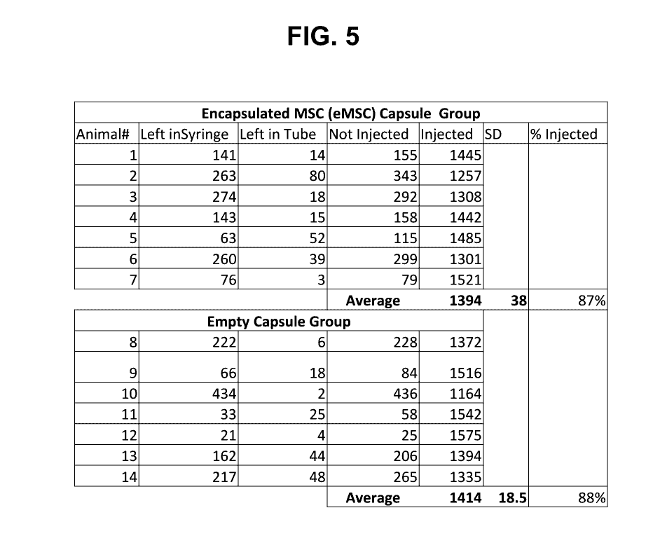

The efficiency and reproducibility of injecting eMSC through 0.8 mm inner diameter thin-walled needles (20 gauge, NIPRO) was analyzed for the IP mouse injections. The 20 gauge needle was used because needles with higher gauge, which have narrower diameters, did not allow reproducible ejection of capsules. We measured the numbers of capsules not injected as the number that remained in the tube and the syringe following IP injections into mice. After injection, the contents of each tube was transferred to a tissue culture dish and the total numbers of capsules were counted using bright field microscopy (this fraction was called “left in tube”). The syringe with the needle attached was washed with saline, transferred to a tissue culture dish and the total numbers of capsules were counted (this fraction was called “left in syringe”). The combined total number of “Not Injected” capsules was subtracted from the total prepared for injection, which was 1600 capsules, and the balance is the calculated number of capsules “injected.” The average “% injected” was calculated as the average number of injected/1600, which represents the efficiency of injection (FIG. 5). SD, standard deviation.

### Example 6

### Treatment of Sepsis with Microcapsules

Sepsis was induced in mice by cecal ligation and puncture (CLP). Adult C57BL/6J mice age of 8-12 weeks were anesthetized with i.p. injection of pentobarbital (50 mg/kg) as described (Csoka et al., FASEB J. 29: 25-36, 2015). Under aseptic conditions, a 2-cm midline laparotomy was performed to allow exposure of the cecum with adjoining intestine. Approximately two thirds of the cecum was tightly ligated with a 3.0 silk suture, and the ligated part of the cecum was perforated twice (through and through) with a 20-gauge needle (BD Biosciences). Thereafter, the ligated cecum was gently squeezed to extrude a small amount of feces through the perforation site and was then returned to the peritoneal cavity. The laparotomy was closed in two layers with 4.0 silk sutures. After the operation, all mice were resuscitated with 1 ml physiological saline injected subcutaneously and returned to their cages with free access to food and water. One hour after CLP, saline or MSC microcapsules were i.p. injected using a thin-walled 20G needle, (inner diameter=0.8 mm, NIPRO) attached to a 1 ml syringe. Sixteen hours after the CLP operation, the mice were re-anesthetized with pentobarbital (50 mg/kg i.p.), and blood and peritoneal lavage were harvested.

### Example 7

### Effects of MSC Microcapsules on Levels of Bacteria in CLP-Induced Sepsis

Microcapsules containing 140,000 live MSC/mouse were i.p. injected into mice one hour followed CLP, and after 16 hours and blood and peritoneal lavage fluid were collected as described in Example 6. Blood or peritoneal lavage fluid was diluted serially in sterile physiologic saline. Fifty microliters of each dilution was aseptically plated and cultured on Trypticase blood agar plates (BD Biosciences) at 37° C. After 24 h of incubation, the number of bacterial colonies was counted. The number of bacteria/culture is expressed as CFUs per milliliter of blood or peritoneal lavage fluid (FIG. 2A-FIG. 2B).

### Example 8

### Effects of MSC Microcapsules on Mouse Cytokines from Septic Mice

Concentrations of IL-10, IL-6, TNFα, IL-1β, MCP-1, and MCP-2 in blood (FIG. 3A-FIG. 3F) and peritoneal lavage fluid (FIG. 4A-FIG. 4F), collected as described in Example 6, were determined using commercially available ELISA kits (R&D Systems, Minneapolis, Minn. USA) according to the manufacturer's instructions.

### Example 9

### Recovery of MSC Microcapsules from Septic Mice

MSC microcapsules were recovered after 16 hours from the peritoneal lavage fluid of CLP injected mice. The fluid was collected in a 1.5 ml microfuge tube and after 2 min the fluid was removed leaving ˜0.05 mL with a precipitate. The precipitate containing microcapsules was washed twice with 1 mL of PBS and resuspended in media. The number of live and dead cells/capsule was measured using the viability assay in Example 3 (FIG. 6A-FIG. 6B).

### Example 10

### Secretion of Human IL-6 from MSC Microcapsules Recovered from CLP Mice

Microcapsules containing ˜5,000 cells maintained in culture or recovered from CLP mice 16 hours after injection as in Example 9 were incubated in media for 24 hours. The media was collected and used to measure Human IL-6 secretion using an Elisa assay for human IL-6 (BioRad) (FIG. 7).

### Example 11

### Secretion of PGE2 from MSC Microcapsules

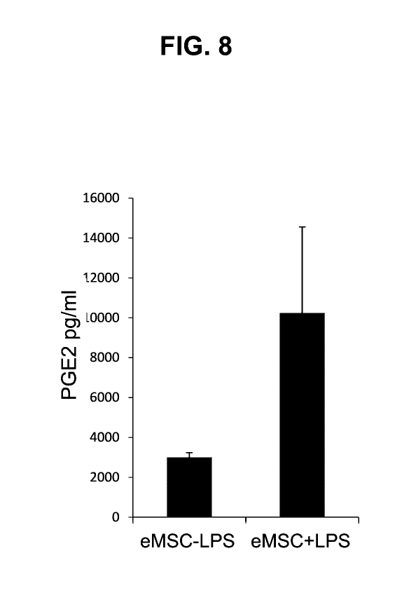

Microcapsules containing ˜5,000 cells maintained in culture were incubated in media for 24 hours. The media was collected and used to measure PGE2 secretion using an Elisa assay for (BioRad) (FIG. 8).

We claim:

1. An isolated population of stem cells wherein said isolated population of encapsulated stem cells secretes at least one therapeutically relevant protein in vivo at least 2 times greater than in vitro.

2. The isolated population of stem cells of claim 1 wherein said stem cells are mesenchymal stem cells (MSCs).

3. The mesenchymal stem cells of claim 2, wherein the mesenchymal stem cells are derived from bone marrow, adipose tissue, umbilical cord, placenta, mesenchymal precursor cells (MPC) or multipotent adult progenitor cells (MAPCs).

4. The isolated population of stem cells of claim 1, wherein the at least one therapeutically relevant protein is selected from the group consisting of epidermal growth factor (EGF), fibroblast growth factor (FGF), platelet-derived growth factor (PDGF), transforming growth factor-B (TGF-B), vascular endothelial growth factor (VEGF), hepatocyte growth factor (HGF), insulin growth factor-1 (IGF-1), angiopoietin-1 (Ang-1), keratinocyte growth factor (KGF), and stromal cell derived factor-1 (SDF-1) and combinations thereof.

5. The isolated population of stem cells of claim 1, wherein the at least one therapeutically relevant protein is selected from the group consisting of Tumor necrosis factor-inducible gene 6 protein (TSG-6), Interleukin 4 (IL-4), Interleukin 5 (IL-5), Interleukin 6 (IL-6), Interleukin 10 (IL-10), Interleukin 33 (IL-33), Interleukin-1 receptor antagonist (IL-1RA), Galectin-1, Galectin-3, adiponectin, resolvin D1 (RvD1) or resolvin E1 (RvE1).

6. The isolated population of stem cells of claim 1, wherein the at least one therapeutically relevant protein is selected from the group consisting of prostaglandins, preferably prostaglandin E2 (PGE2).

7. The isolated population of stem cells of claim 1, wherein the therapeutic effect of said encapsulated stem cells is to repair injured tissue caused by a disease, to slow progression of a disease, or to relieve symptoms of a disease.

8. The disease of claim 7, wherein said disease is selected from Sepsis, Acute Lung Injury (ALI), Acute respiratory distress syndrome (ARDS), Critical Limb Ischemia (CLI), Spinal Cord Injury (SCI), Traumatic Brain Injury (TBI), Ebola, Acute Lung Injury (ALI), and Acute Respiratory Distress Syndrome (ARDS).

9. The disease of claim 7, wherein said disease is selected from the list of Inflammatory bowel disease (IBD), Crohn's disease, Rheumatoid arthritis (RA), Congestive Heart Failure, Amyotrophic Lateral Sclerosis (ALS), Diabetic Retinopathy (DR), Macular Degeneration (MD), Parkinson's Disease (PD), Multiple Sclerosis (MS), Type 1 Diabetes and Type 2 Diabetes.

10. An isolated population of stem cells comprising an exogeneous DNA sequence expressing an siRNA, miRNA, or dsRNA molecule, wherein the encapsulated stem cell delivers the siRNA, miRNA, or dsRNA polynucleotide to a target cell via a microvesicle, exosome, or a cellular protrusion.

11. An isolated population of stem cells of claim 10, wherein the isolated population of encapsulated stem cells are in communication with a target cell under conditions suitable for transfer of the siRNA, miRNA, or dsRNA polynucleotide to a target cell via a microvesicle, exosome, or a cellular protrusion.

12. An isolated population of stem cells of claim 11, wherein the isolated population of encapsulated stem cells delivers the exogenous DNA sequence or the siRNA, miRNA, or dsRNA sequence by a microvesicle, exosome or a cellular protrusion.

13. An isolated population of stem cells of claim 12, wherein the siRNA, miRNA, or dsRNA is directed at a gene mediating a viral infection.

14. The viral infection of claim 13, is caused by the Ebola virus.

15. The isolated population of stem cells of claim 13, wherein the siRNA, miRNA, or dsRNA is directed at the NPC1 receptor gene.

16. An isolated population of stem cells of claim 13 which is an expanded clonal population of mesenchymal stem cells.

17. A micro-encapsulation system comprising an alginate microcapsule, wherein the micro-encapsulation system is capable of immobilizing the isolated population of stem cells of claim 1 within an alginate microenvironment while sustaining molecular communication to relieve disease or its symptoms in an animal or human subject.

18. The micro-encapsulation system of claim 17, wherein the alginate polymer has a concentration in the range from about 1.0% (w/v) to about 3% (w/v).

19. The micro-encapsulation system of claim 18, wherein the alginate polymer has a concentration of about 2.5% (w/v).

20. The micro-encapsulation system of claim 17, wherein the microcapsule comprises an additional sequential external surface coating of poly-L-lysine.

21. The micro-encapsulation system of claim 17, wherein the microcapsule comprises a divalent cation from the group of calcium or barium or a mixture of calcium and barium to crosslink the alginate polymer into a microcapsule.

22. The micro-encapsulation system of claim 17, wherein the divalent cation is mixture of 50-100 mM calcium and 2-50 mM barium, preferably 50 mM calcium and 50 mM barium.

23. The micro-encapsulation system of claim 22, wherein the microcapsule is highly permeable to albumin but not to immunoglobulin G (IgG).

24. A method of treating a disease comprising administering to a subject suffering from a disease an effective amount of stem cells immobilized in a micro-encapsulation system as recited in claim 17 to regulate an immune response in said subject to relieve a disease.

25. The method of claim 24, wherein the dose of stem cells administered to a subject is less than 10 million cells/kg weight of the subject.

26. The method of claim 24, wherein the dose of stem cells administered to a subject is approximately 6 million cells/kg weight of the subject.

27. The method of claim 24, wherein an effective amount of stem cells immobilized in a micro-encapsulation system are administered to a subject by intravenous injection, intraperitoneal injection, lymph node injection, thymus injection, spleen injection, subcutaneous injection or combinations thereof.

28. The method of claim 27, wherein an effective amount of stem cells are administered within 1 day of diagnosis of a subject in need of treatment for a disease or condition.

29. The method of claim 28, wherein an effective amount of stem cells are administered within 1 day of diagnosis of a subject in need of treatment followed by a second effective amount of stem cells administered 2-7 days later.

30. The method of claim 24, wherein the stem cells are derived from a non-autologous subject other than the subject in need of treatment for a disease.

31. The method of claim 24, wherein an effective amount of stem cells are administered to increase macrophage phagocytosis of bacteria in a subject with a disease involving increased levels of bacteria in blood or peripheral tissues.

32. The method of claim 24, wherein the disease is sepsis, severe sepsis, or septic shock.

33. The method of claim 27, wherein an effective amount of stem cells are administered to decrease levels of bacteria in the blood or peritoneum of a subject with a disease by at least 100-fold.
